(19) United States
(12) Patent Application Publication
(10) Pub. No.: US 2016/0081675 A1
(43) Pub. Date: Mar. 24, 2016

(54) HELICAL DRIVEN ROTATING TISSUE COLLECTION

(71) Applicant: Boston Scientific Scimed, Inc., Maple Grove, MN (US)

(72) Inventors: John B. Golden, Norton, MA (US); Kirsten Viering, Watertown, MA (US); Matthew Donovan, Raynham, MA (US); Eric M. Schneider, Lincoln, RI (US); Dylan Murphy, Walpole, MA (US); Michael McBrien, Marlborough, MA (US); Mahfuza Ahmed, Brookline, MA (US); Amie Preston, Groton, MA (US); Daniel R. Quinn, Littleton, MA (US)

(21) Appl. No.: 14/857,244

(22) Filed: Sep. 17, 2015

Related U.S. Application Data
(60) Provisional application No. 62/052,166, filed on Sep. 18, 2014.

(51) Int. Cl. A61B 10/02

(52) U.S. Cl. CPC A61B 10/0266; A61B 10/0283; A61B 2010/0208

(57) ABSTRACT

A device for collecting a tissue sample includes an outer sheath extending from a proximal end to a distal end and including a lumen extending therethrough. The device also includes a needle movably housed within the outer sheath. The needle extends from a proximal end to a serrated distal end and including a channel extending therethrough. The needle is longitudinally movable relative to the outer sheath between an insertion configuration, in which the distal end of the needle is proximal of the distal end of the outer sheath, and a tissue collecting configuration, in which the distal end of the needle extends distally past the distal end of the outer sheath to penetrate tissue and collect a tissue sample in the channel. In addition, the device includes a drive mechanism rotating a distal portion of the needle about a longitudinal axis thereof as the needle is moved between the insertion and tissue collecting configurations.

### PRIORITY CLAIM

The present disclosure claims priority to U.S. Provisional Patent Application Ser. No. 62/052,166 filed Sep. 18, 2014; the disclosure of which is incorporated herewith by reference.

### BACKGROUND

Needle biopsy procedures are common for the diagnosis and the staging of disease. In particular, in endoscopic ultrasound-guided fine needle aspiration (EUS-FNA), the needle is advanced under ultrasound guidance so that the physician may visualize a position of the needle relative to target tissue. A distal end of the needle is then inserted into the target tissue mass to collect a sample of the tissue in a lumen thereof. Thus, EUS-FNA ensures that the correct tissue is sampled while minimizing risk to the patient. Although EUS-FNA is a highly sensitive and specific procedure, it may be difficult to acquire a suitable sample under certain clinical situations. The more cells or tissue that can be acquired, in particular for histological samples, the greater the potential for a definitive diagnosis. Larger gauge needles, however, may be difficult to pass through tortuous anatomy and may acquire samples including more blood, making it more difficult to obtain a diagnosis.

### SUMMARY

The present disclosure relates to a device for collecting a tissue sample, comprising an outer sheath extending longitudinally from a proximal end to a distal end and including a lumen extending therethrough along with a needle movably housed within the outer sheath, the needle extending longitudinally from a proximal end to a serrated distal end and including a channel extending therethrough, the needle being longitudinally movable relative to the outer sheath between an insertion configuration, in which the distal end of the needle is proximal of the distal end of the outer sheath, and a tissue collecting configuration, in which the distal end of the needle extends distally past the distal end of the outer sheath to penetrate target tissue and to collect a tissue sample in the channel and a drive mechanism rotating a distal portion of the needle about a longitudinal axis thereof as the needle is moved between the insertion configuration and the tissue collecting configuration.

In an embodiment, the drive mechanism may include a helical structure extending about the distal portion of the needle and a corresponding helical structure along an interior surface of a drive collar connected to the distal end of the outer sheath, the helical structure of the needle and the corresponding helical structure of the drive collar engaging one another such that, when the needle is moved longitudinally relative to the outer sheath, the needle is rotated relative to the outer sheath.

In an embodiment, the needle may include a barb extending laterally into the channel to grip tissue received therewithin.

In an embodiment, the barb may be a tab formed by cutting through a wall of the needle and bending the tab laterally into the channel.

In an embodiment, the drive mechanism may include a first ratchet mechanism and a second ratchet mechanism coupling a proximal portion and a distal portion of the needle, the first ratchet mechanism including a rod extending distally from the proximal portion to engage ratchet teeth along an interior surface of the distal portion, and the second ratchet mechanism including a set of teeth about each of a distal end of the proximal portion and a proximal end of the distal portion such that teeth of the first and second ratchet mechanisms are alternatingly engaged to rotate the distal portion relative to the proximal portion.

In an embodiment, the first and second ratchet mechanisms may be alternatingly engaged via a linear oscillation of the proximal portion.

In an embodiment, the device may further comprise a stylet including a protrusion extending laterally therefrom.

In an embodiment, the drive mechanism may include a ratchet mechanism coupling a proximal portion and a distal portion of the needle, the ratchet mechanism including a groove extending along an interior surface of the distal portion of the needle, the protrusion of the stylet engaging ratchet teeth along the groove so that a linear oscillation of the stylet rotates the distal portion relative to the proximal portion.

In an embodiment, the needle may include a proximal portion and a distal portion connected to one another via a pivot joint controlled via a plurality of control wires to pivot the distal portion relative to the proximal portion.

In an embodiment, the needle may include a plurality of holes extending laterally through a distal portion thereof for permitting fluid to leak therethrough as tissue is being cut.

In an embodiment, the device may further comprise a suction source applying a suction force through the channel of the needle to draw tissue thereinto.

The present disclosure also relates to a device for collecting a tissue sample, comprising an outer sheath extending longitudinally from a proximal end to a distal end and including a lumen extending therethrough and a needle slidably housed within the outer sheath, the needle extending longitudinally from a proximal end to a tapered distal end, the needle longitudinally movable relative to the outer sheath between a retracted configuration and a tissue collecting configuration in which the needle is moved distally relative to the outer sheath.

In an embodiment, a tissue sample may be collected in a space between an interior surface of the lumen of the outer sheath and an exterior surface of the needle.

In an embodiment, a suction force may be applied through the space to draw tissue thereinto.

In an embodiment, the needle may be moved between the retracted and tissue collecting configurations via a drive mechanism including a first cam attached to the proximal end of the needle, an second cam housed within a proximal end of the outer sheath and a spring element biasing the device toward the retracted configuration.

The present disclosure is also directed to a method for collecting a tissue sample, comprising inserting a tissue collecting device to a target tissue within a patient body via a working channel of an endoscope, in an insertion configuration in which a needle is housed within an outer sheath so that a distal end of the needle is proximal a distal end of the outer sheath and moving the needle distally relative to the outer sheath to a tissue collecting configuration in which the distal end of the needle extends distally beyond the distal end of the outer sheath to be inserted into the target tissue, wherein the needle rotates about a longitudinal axis thereof via a drive mechanism as the needle is advanced distally out of the sheath such that a serrated distal edge of the needle cores a tissue sample from the target tissue collecting the tissue sample therewithin.

In an embodiment, the method may further comprise moving the needle from the tissue collecting position to the retracted position to withdraw the device from the patient body.

In an embodiment, the drive mechanism may include a helical structure extending about a distal portion thereof and a corresponding helical structure along an interior surface of a drive collar connected to the distal end of the outer sheath, the helical structure of the needle and the corresponding helical of the outer sheath engaging one another such that, when the needle is moved longitudinally relative to the outer sheath, the needle is rotated relative to the outer sheath.

In an embodiment, the drive mechanism may include a first ratchet mechanism and a second ratchet mechanism coupling a proximal portion and a distal portion of the needle, the first ratchet mechanism including a rod extending distally from the proximal portion to engage ratchet teeth along an interior surface of the distal portion, and the second ratchet mechanism including a set of teeth about each of a distal end of the proximal portion and a proximal end of the distal portion such that teeth of the first and second ratchet mechanisms are alternatingly engaged to rotate the distal portion relative to the proximal portion.

In an embodiment, the drive mechanism may include a ratchet mechanism coupling a proximal portion and a distal portion of the needle, the ratchet mechanism including a groove extending along an interior surface of the distal portion of the needle to receive a protrusion extending laterally from a portion of a stylet received within the channel, the protrusion of the stylet engaging ratchet teeth along the groove so that a linear oscillation of the stylet rotates the distal portion relative to the proximal portion

### BRIEF DESCRIPTION

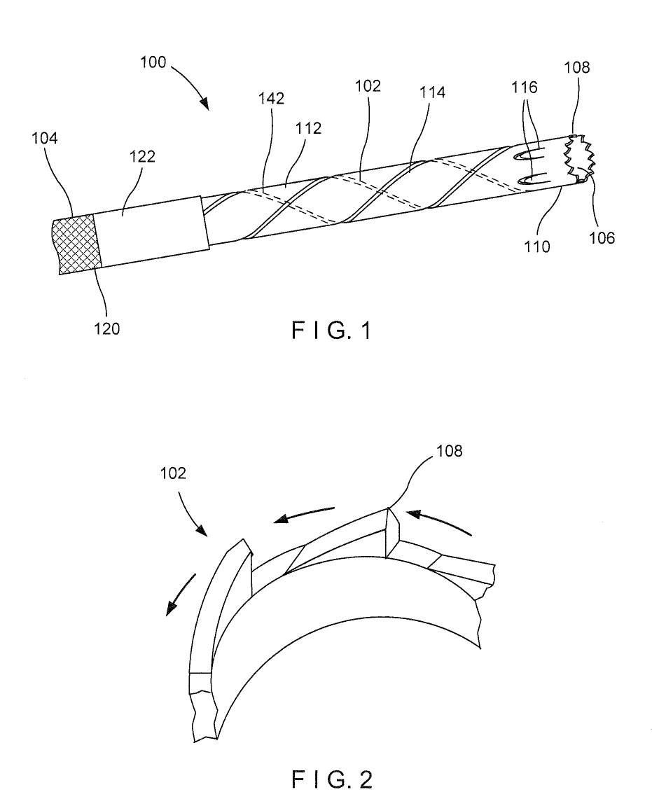

FIG. 1 shows a perspective view of a device according to an exemplary embodiment of the present disclosure;

FIG. 2 shows a perspective view of a portion of a distal cutting edge of the device of FIG. 1;

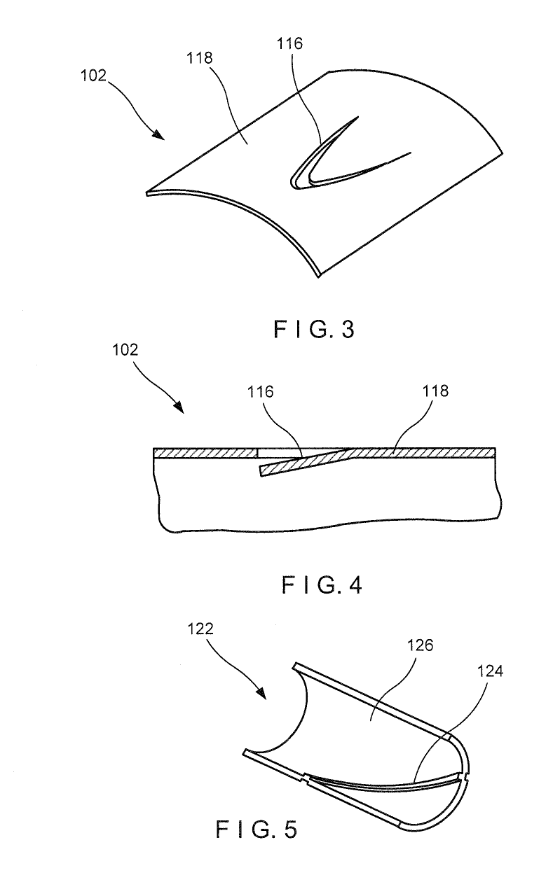

FIG. 3 shows a perspective view of a portion of a needle of the device of FIG. 1 including an interior barb;

FIG. 4 shows a cross-sectional view of the portion of the needle of FIG. 3 including the interior barb;

FIG. 5 shows a perspective view of a portion of a drive collar of the device of FIG. 1;

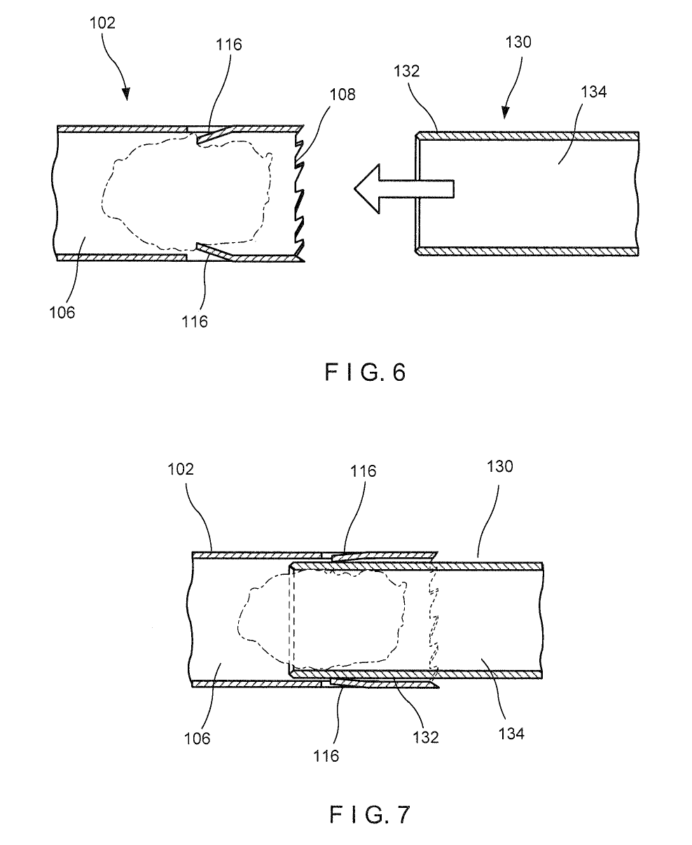

FIG. 6 shows a cross-sectional view of a sample removal tool for the device of FIG. 1, in a first position;

FIG. 7 shows a cross-sectional view of the sample removal tool of FIG. 6, in a second position;

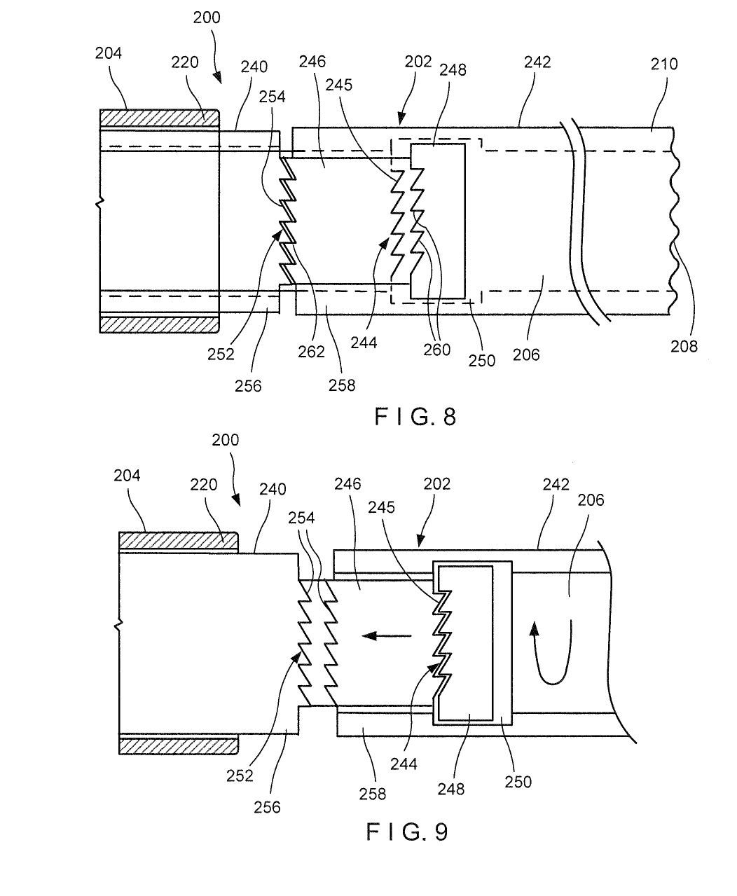

FIG. 8 shows a longitudinal cross-sectional view of a device according to another exemplary embodiment of the present disclosure, in a first configuration;

FIG. 9 shows a longitudinal cross-sectional view of the device of FIG. 8, in a second configuration;

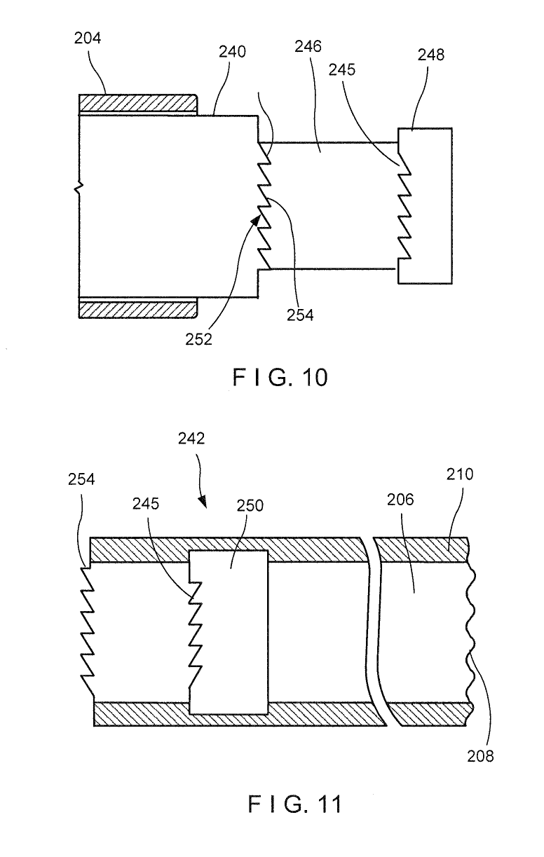

FIG. 10 shows a longitudinal cross-sectional view of proximal portion of a needle of the device of FIG. 8;

FIG. 11 shows a longitudinal cross-sectional view of a distal portion of the needle of the device of FIG. 8;

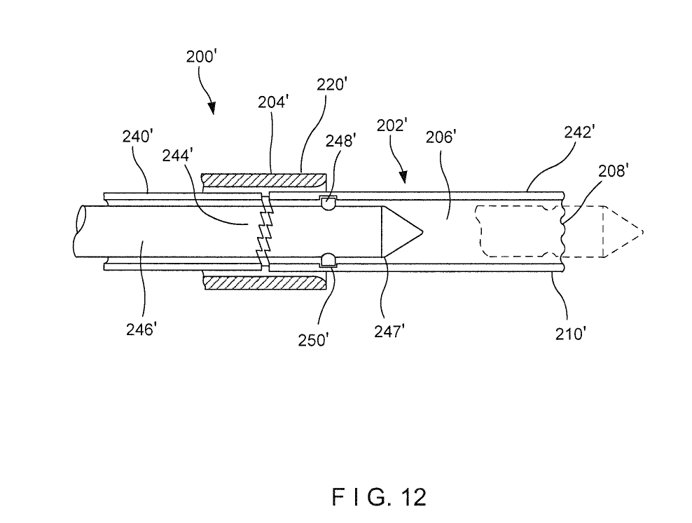

FIG. 12 shows a longitudinal cross-sectional view of a device according to yet another exemplary embodiment of the present disclosure;

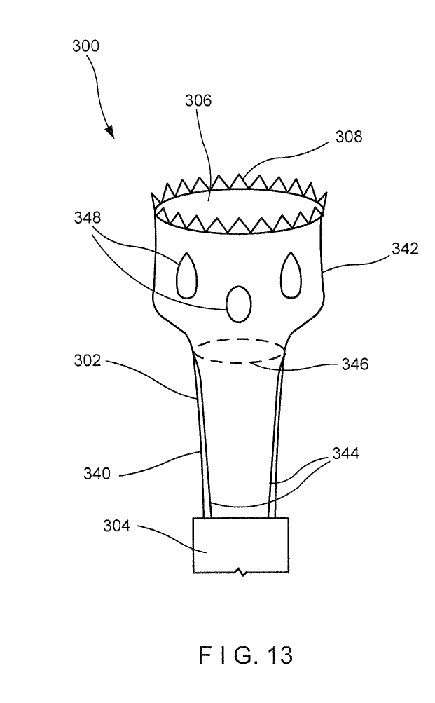

FIG. 13 shows a side perspective view of a device according to another exemplary embodiment of the present disclosure;

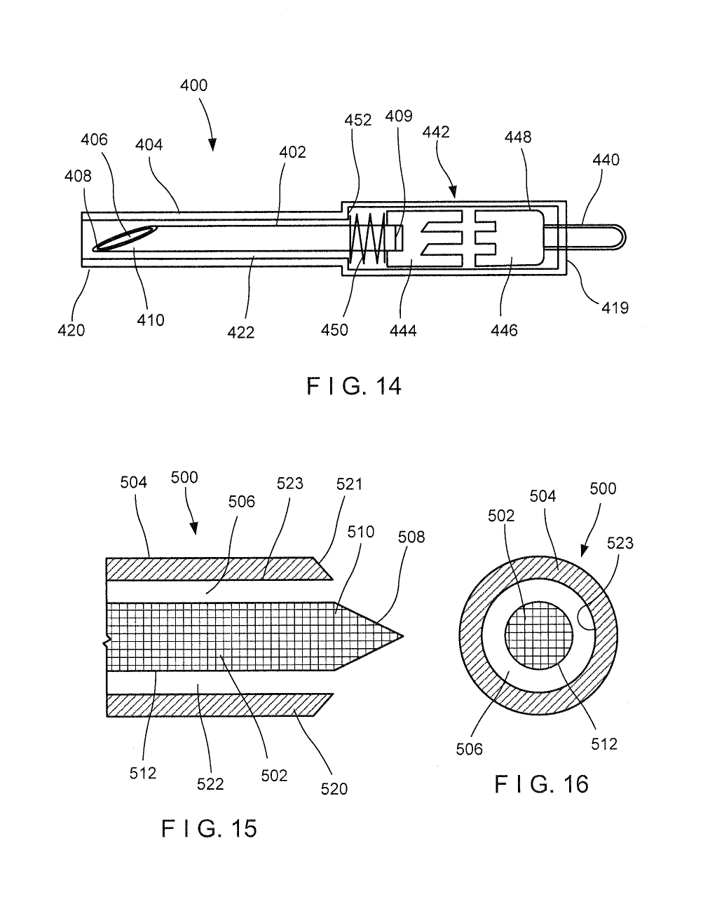

FIG. 14 shows a transparent side view of a device according to yet another exemplary embodiment of the present disclosure;

FIG. 15 shows a longitudinal cross-sectional view of a distal portion of a device according to another exemplary embodiment of the present disclosure; and

FIG. 16 shows a lateral cross-sectional view of the device of FIG. 15.

### DETAILED DESCRIPTION

The present disclosure may be further understood with reference to the following description and the appended drawings, wherein like elements are referred to with the same reference numerals. The present disclosure is related to devices for obtaining tissue samples and, in particular, EUS-FNA devices. Exemplary embodiments of the present disclosure describe devices comprising needles which rotate about a longitudinal axis thereof and which may include a sharp or serrated distal edge permitting a tissue sample to be cored and collected within a channel thereof. It should be noted that the terms “proximal” and “distal” as used herein, are intended to refer to a direction toward (proximal) and away from (distal) a user of the device.

As shown in FIGS. 1-7, a device 100 according to a first exemplary embodiment of the present disclosure comprises a needle 102 housed within an outer sheath 104 for movement between an insertion configuration and a tissue collecting configuration. In the insertion configuration, a distal end 110 of the needle 102 is received within the sheath 104 and does not extend distally past a distal end 120 of the sheath 104. In the tissue collecting configuration, the needle 102 is moved distally relative to the sheath 104 such that the needle 102 rotates about a longitudinal axis thereof, in a first direction, as the needle 102 is driven longitudinally out of the sheath 104 so that the distal end 110 of the needle 102 extends distally past the distal end 120 of the sheath 104 for penetration of target tissue. As the needle 102 is rotated out of the sheath 104, a serrated distal edge 108 of the needle 102 cores a sample from the target tissue, collecting the tissue sample within a channel 106 thereof. Methods utilizing current existing EUS-FNA needles generally involve repeated insertion of the needle distally into a tissue mass to separate the tissue sample from the surrounding tissue mass. In some cases, however, this methodology produces tissue samples or histology with significant blood contamination, preventing an accurate diagnosis. The rotation of the serrated distal edge 108 of the needle 102 minimizes trauma to the surrounding tissue and, therefore, minimizes blood contamination, enabling the collection of a better sample. Once a tissue sample has been collected within the needle 102, the needle 102 is retracted back into the sheath 104. Drawing the needle 102 proximally relative to the sheath 104 rotates the needle 102 the longitudinal axis in a second direction opposite the first direction. The device 100 may further comprise a handle assembly (not shown) coupled to proximal ends of the sheath 104 so that it remains exterior to the body while the distal portion of the device 100 is inserted into the body to a target tissue site. As would be understood by those skilled in the art, the handle assembly may include an actuator(s) for controlling the movement of the needle 102 relative to the sheath 104.

The needle 102 extends longitudinally from a proximal end (not shown) to a distal end 110 and includes the channel 106 extending therethrough. In one embodiment, the needle 102 may be comprised of a longitudinally extending proximal portion (not shown) and a longitudinally extending distal portion 142 connected to one another such that the distal portion 142 is rotatable about the longitudinal axis relative to the proximal portion. An exterior surface 112 of the distal portion 142 of the needle 102 includes a helical structure 114 extending about a length thereof for driving the needle 102 rotationally in and out of the sheath 104. In one embodiment, the helical structure 114 is formed as a helical recess in the exterior surface 112 which engages a corresponding structure on an inner surface of the distal end of the sheath 104 so that, as the needle 102 is advanced longitudinally through the sheath 104, a portion of the longitudinal motion is converted to rotary motion. For example, the structure may include a protrusion extending radially inward from an inner surface of the sheath 104 to engage the helical structure 114 (recess) to rotate the needle 102 as it moves along or past the protrusion. The protrusion may extend along a helical path corresponding to the helical recess or may simply engage a short portion of this recess. Similarly, the needle 102 may include only a structure which is longitudinally short and which rides in or which receives an elongated helical structure formed on an inner surface of the sheath 104.

In another embodiment, the helical structure 114 may be a helically shaped protrusion extending radially outward from the exterior surface 112. The helical structure 114 extends about a distal portion 142 of the needle 102 along a length corresponding to a desired insertion length of the needle 102 into the target tissue and may engage a corresponding groove or recess formed on an inner surface of the sheath 104. For example, the helical structure 114 may extend along a length of the distal portion 142 of the needle 102 selected to enable the collection of a core tissue sample up to 2 cm in length. It will be understood by those of skill in the art, however, that a length of the helical structure 114 may be varied to collect a larger or smaller core tissue sample, as desired.

As shown in FIG. 2, the distal edge 108 at the distal end 110 is serrated so that, when the needle 102 is rotated about the longitudinal axis thereof, the distal edge 108 cuts into target tissue which it contacts. As shown in FIGS. 3-4, the needle 102 may also include one or more internal barbs 116 extending into the channel 106 for trapping or holding the tissue sample therewithin. The internal barbs 116 may be formed by, for example, stamping or laser cutting a portion of a wall 118 defining the channel 106 to form tabs bent radially into the channel 106 and pointing toward the proximal end of the needle 102 so that the tissue sample may be easily received proximally into the channel 106, but prevented from slipping distally out of the channel 106. The barbs 116 may be biased toward the radially inward position but formed of a material which permits the barbs 116 to be moved to a radially outward position in which the barbs 116 do not extend into the channel 106.

The sheath 104 extends longitudinally from a proximal end (not shown) to the distal end 120 and includes a lumen extending therethrough. The lumen is sized and shaped to slidably receive the needle 102 therein. In particular, the needle 102 is rotatable and longitudinally movable within the lumen of the sheath 104. The distal end 120 of the sheath 104 includes a drive collar 122 extending distally therefrom and including a lumen axially aligned with the lumen of the sheath 104 for receiving the needle 102 therethrough.

As shown in FIG. 5, the drive collar 122 includes a structure 124 along an interior surface 126 corresponding to the helical structure 114 of the needle 102. For example, where the helical structure 114 is a helically shaped recess about the exterior surface 112 of the needle 102, the corresponding helical structure 124 is a corresponding helical protrusion along the interior surface 126 of the drive collar. Where the helical structure 114 is a helically shaped protrusion extending from the exterior surface 112, the corresponding helical structure 124 may be a corresponding helical recess along the interior surface 126. The corresponding helical structure 124 permits the needle 102 to be rotated and moved longitudinally relative to the sheath 104. Thus, the helical structure 114 and the corresponding helical structure 124 are configured to permit slidable movement relative to one another.

According to an exemplary method using the device 100, the device 100 may be inserted to a target tissue within a patient's body via, for example, a working channel of an endoscope. To prevent damage to the working channel and to non-targeted tissue, the device 100 is inserted through the working channel in the insertion configuration with the distal edge 108 of the needle 102 received within the sheath 104. Once the device 100 has reached the target tissue site, the needle 102 is driven longitudinally distally so that it is driven distally out of the sheath 104 and rotated about the longitudinal axis relative to the sheath 104 in the first direction into the target tissue. In particular, as the needle 102 is moved distally relative to the sheath 104, the distal portion 142 of the needle 102 rotates about the longitudinal axis via the engagement between the helical structure 114 and the corresponding helical structure 124 of the sheath 104. Rotation of the distal portion 142 of the needle 102 as it is driven distally out of the sheath 104 causes the serrated distal edge 108 to core a tissue sample from the target tissue and to receive and trap the tissue sample within the channel 106. The tissue sample is moved proximally into the channel 106 beyond the barbs 116 so that it is held therein. Once the desired tissue sample has been received within the channel 106, the needle 102 may be retracted into the sheath 104 by drawing the needle 102 proximally. As the needle 102 is drawn proximally, the helical structure 114 interfaces with the corresponding helical structure 124 to rotate the needle 102 in the second direction opposite the first direction. The barbs 116 traps and hold the tissue sample within the channel 106 as the needle 102 is retracted proximally into the sheath 102.

As shown in FIGS. 6 and 7, upon removal of the device 100 from the target tissue, the tissue sample may be removed from the channel 106 using, for example, a removal tool 130 which moves the barbs 116 from the radially inward position to the radially outward position so that the barbs 116 no longer engage the tissue sample collected within the channel 106. The removal tool 130 may include, for example, a tubular element having an outer diameter slightly smaller than an inner diameter of the needle 102. Thus, when the removal tool 130 is inserted proximally into the channel 106, an exterior surface 132 of the removal tool 130 contacts the barbs 116 moving the barbs 116 radially outward to disengage the collected tissue sample. The tissue sample may then be removed from the channel 106 via a lumen 134 of the removal tool 130 using known methods such as, for example, sample flushing.

Although a stylet is not explicitly shown or described, it will be understood by those of skill in the art that the device 100 may further comprise a stylet. The stylet may be received within the channel 106 of the needle 102 during insertion of the device 100 into the body to prevent non-targeted tissue from entering the channel 106. Once the device 100 has reached the target tissue site, the stylet may be removed therefrom to facilitate collection of the tissue sample within the channel 106.

As shown in FIGS. 8-11, a device 200 is similar to the device 100 described above, comprising a needle 202 movably housed within a sheath 204 so that the needle 202 is rotatable relative to the sheath 204 about a longitudinal axis thereof between an insertion configuration, in which a distal end 210 of the needle 202 is proximal of a distal end 220 of the sheath 204, and a tissue collecting configuration in which the distal end 210 of the needle 202 extends distally past the distal end 220 of the sheath 204 into a target tissue. The needle 202 is also substantially similar to the needle 102, described above, including a serrated distal edge 208 which cores a tissue sample from a target tissue, in which the needle 202 is inserted, via rotation of the needle 202 about the longitudinal axis, so that the tissue sample is collected in a channel 206 thereof.

The needle 202, however, includes a proximal portion 240 and a distal portion 242 connected to one another via a first ratchet mechanism 244 including a rod 246 rigidly coupled to the proximal portion 240 and which includes an engaging end 248 slidably received in a groove 250 formed on an inner surface of the distal portion 242. Each of the groove 250 and the engaging end 248 include a set of teeth 245 thereabout, which selectively engage one another to control rotation of the distal portion 242 relative to the proximal portion 240. The needle 202 further includes a second ratchet mechanism 252 for controlling the rotation of the distal portion 242 relative to the proximal portion. The second ratchet mechanism 252 includes a set of teeth 254 on each of a distal end 256 of the proximal portion 240 and a proximal end 258 of the distal portion 242.

A length of the groove 250 is longer than a length of the engaging end 248 so that the engaging end 248 is movable therein between a proximal position and distal position. When the rod 246 is advanced distally, as shown in FIG. 8, the teeth 254 of the second ratchet mechanism 252 are engaged while the teeth 245 of the first ratchet mechanism 244 are disengaged. When the rod 246 is partially retracted relative to the proximal portion 240, as shown in FIG. 9, the teeth 254 of the second ratchet mechanism 252 disengage while the teeth 245 of the first ratchet mechanism 244 are engaged. Ramped surfaces 260, 262 of each of the set of teeth 245, 254, respectively, extend in opposite directions so that the alternating engagement and disengagement of the teeth 245, 254 of the first and second ratchet mechanisms 244, 252, respectively, causes the distal portion 242 of the needle 202 to rotate about the rod 246 relative to the proximal portion 240. The advancement and partial retraction of the rod 246 may be achieved via a linear oscillation facilitated by an oscillating input at the proximal end of the needle 202. Thus, the linear oscillation of the rod 246 may result in a continuous rotation of the distal portion 244 of the needle 202 relative to the rod 246 and the proximal portion 240.

As shown in FIG. 12, a device 200′ according to an alternate embodiment of the present disclosure is substantially similar to the device 200 described above, comprising a needle 202′ movably housed within a sheath 204′ so that the needle 202′ is rotatable relative to the sheath 204′ about a longitudinal axis thereof between an insertion configuration, in which a distal end 210′ of the needle 202′ is proximal of a distal end 220′ of the sheath 204′, and a tissue collecting configuration in which the distal end 210′ of the needle 202′ extends distally past the distal end 220′ of the sheath 204′ into a target tissue. Similarly to the needle 202, the needle 202′ includes a proximal portion 240′ and a distal portion 242′ rotatable relative to one another about a longitudinal axis thereof via, for example, a ratchet mechanism. Rather than a rod oscillated via an oscillating unit at a proximal end thereof, however, the device 200′ includes a linearly oscillating stylet 246′ received within a channel 206′ of the needle 202′. In an insertion configuration (shown in phantom), the stylet 246′ is received within the needle 202′ such that a distal tip 247′ thereof is slightly distal of the distal end 210′ of the needle 202′. In a tissue-collecting configuration, the stylet 246′ is drawn proximally relative to the needle 202′ until a protrusion 248′ extending laterally therefrom is received within a groove 250′ extending about an interior of the needle 202′. Thus, tissue may be cored via a distal serrated edge 208′ and collected within a portion of the channel 206′ distal of the distal tip 247′ of the stylet 246′.

The groove 250′ formed on an inner surface of the distal portion 242′. The groove 250′ includes a series of projecting portions each of which extends along a portion of a helix so that as the stylet 246′ is advanced distally relative to the distal portion 242′, the engaging portion 248 rides in a helical portion of the groove 250′ to rotate the distal portion 242′ relative to the proximal portion 240′ through an angle corresponding to a portion of the circumference of the distal portion 242′ corresponding to a width of a single tooth so that, as the distal portion 242′ is rotated over a single tooth of the ratchet mechanism 244′ along the proximal portion 240′, the subsequent tooth engages the distal portion 242 to prevent its rotating in the opposite direction back to its original position. Then, when the stylet 246′ is withdrawn proximally until the protrusion 248′ reaches a proximal end of the helical portion of the groove 250′, the protrusion 248′ rotates through a circumferential portion of the groove 250′ under its natural bias to reach a second helical portion of the groove 250′ so that as the process is repeated, each distal advancement of the stylet 246′ rotates the distal portion 242′ relative to the proximal portion 240′ by an amount corresponding to the width of one of the teeth.

As shown in FIG. 13, a device 300 according to another exemplary embodiment of the present disclosure may be substantially similar to the devices 100, 200, described above, comprising a needle 302 rotatably housed within an outer sheath 304 between an insertion configuration and a tissue collecting configuration. The needle 302 includes a proximal portion 340 and a distal tissue cutting portion 342. The needle 302 is configured so that rotation of the proximal portion 340 about a longitudinal axis thereof translates to a corresponding rotational movement of the distal portion 342. The proximal and distal portions 340, 342, however, are also connected to one another via a pivot joint 346. In particular, control wires 344 may extend from the distal portion 342 to a proximal end of the device 300 which may include, for example, a handle assembly including actuators for controlling the control wires 344. The control wires 344 may pivot the distal portion 342 of the needle 302 relative to the proximal portion 342 in, for example, four directions to provide leverage to cut more tissue.

The distal portion 342 includes a distal edge 308 that may be substantially similar to the distal edge 108 of the needle 102. In particular, the distal edge 308 may include serrations which, when the needle 302 is advanced distally into the target tissue via a rotation thereof about the longitudinal axis, core a tissue sample from the target tissue into which it is inserted, collecting the tissue sample in a channel 306 thereof. The distal portion 342 may also include a plurality of holes 348 extending laterally through a wall thereof. The holes 348 may be particularly configured to allow for some fluid to leak therepast during the cutting of the target tissue.

The device 300 may further comprise a suction source for applying a suction force through the channel 306 of the needle 302 to suction tissue into the channel 306, holding the tissue therein during the cutting thereof. Once the tissue sample has been cored from the target tissue, the target tissue is held within the channel 306 during removal of the device 300 from the patient body.

As shown in FIG. 14, a device 400 according to another exemplary embodiment of the present disclosure may be substantially similar to the device 100, comprising a needle 402 movably housed within an outer sheath 404. Rather than being rotated relative to the outer sheath 404 about a longitudinal axis thereof, however, the needle 402 is inserted into a target tissue to collect a tissue sample within a channel 406 thereof via a sudden, precise insertion in the target tissue and a quick retraction. The quick insertion and retraction of the needle 402 is actuated via a single motion of a pin 440 at a proximal end of the device 400. Pushing the pin 440 distally with respect to the outer sheath 404 actuates a drive mechanism 442 which drives the quick insertion/retraction of the needle 402 relative to the outer sheath 404. The quick insertion/retraction of the needle 402 into the target tissue may permit the collection of a larger tissue sample.

The needle 402 extends longitudinally from a proximal end 409 to a distal end 410 and includes a channel 406 extending therethrough for collecting the tissue sample therein. The distal end 410 may include a beveled or tapered tip 408 for piercing the target tissue into which the needle 402 is inserted. The outer sheath 404 extends longitudinally from a proximal end 419 to a distal end 420 and includes a lumen 422 extending therethrough for slidably receiving the needle 402 therein. In a retracted position, the needle 402 is housed within the outer sheath 404 such that the distal end 410 of the needle 402 is proximal of the distal end 420 of the outer sheath 404. In a tissue insertion position, the distal end 410 of the needle 402 extends distally past the distal end 420 of the outer sheath 404 to pierce the target tissue with the tapered tip 408, coring and collecting the target tissue within the channel 406.

The drive mechanism 442 includes a first cam 444 connected to the proximal end 409 of the needle 402 and a second cam 446 housed within the lumen 422 at the proximal end 419 of the outer sheath 404. A proximal end 448 of the second cam 446 is connected to the pin 440, which extends proximally past the proximal end 419 of the outer sheath 404 to be accessible by a user of the device 400. The drive mechanism 442 also includes a spring element 450 housed within the outer sheath 404 distally of the first cam 444, between the first cam 444 and a shoulder 452 extending radially into the lumen 422.

When the pin 440 is pushed distally relative to the outer sheath 404, the second cam 446 moves distally to interface with the first cam 442, pushing the needle 402 distally out of the outer sheath 404 from the retracted position to the tissue penetration position. The distal movement of the first and second cams 444, 446 relative to the outer sheath 404 also causes the spring element 450 distal of the first cam 444 to become compressed so that, upon release of the pin 440 by the user, the spring element 450 is released to revert to its biased configuration, moving the first cam 444 and the needle 402 proximally relative to the outer sheath 404, into the retracted position. Thus, a simple press and release of the pin 440 inserts and retracts the needle 402, allowing a tissue sample to be collected into the channel 406 in a single motion.

As shown in FIGS. 15-16, a device 500 according to another exemplary embodiment of the present disclosure is substantially similar to the device 400 described above, comprising a needle 502 slidably housed within an outer sheath 504. Similarly to the needle 402, the needle 502 extends longitudinally from a proximal end (not shown) to a distal end 510 including a tapered tip 508. The outer sheath 504 extends from a proximal end (not shown) to a distal end 520 and includes a lumen 522 extending therethrough. The lumen 522 is sized and shaped so that when the needle 502 is received therein, a space 506 between an interior surface 523 of the lumen 522 and an exterior surface 512 of the needle 502 is configured to receive a tissue sample therein. The distal end 520 of the outer sheath 504 includes a beveled edge 521.

In an insertion configuration, the tapered tip 508 of the needle 502 extends slightly distally beyond the beveled edge 521 of the outer sheath 504 such that the tapered tip 508 and beveled edge 521 together permit seamless insertion of the device 500 into target tissue. Once the device 500 has been inserted into the target tissue, the needle 502 is moved distally relative to the outer sheath 504, causing portions of the target tissue to be moved into the space 506 between the outer sheath 504 and the needle 502. A suction force may be applied through the space 506 to draw the target tissue into the space 506. The radial pressure of the target tissue on the beveled edge 521 of the outer sheath 504 when the needle 502 is moved distally relative to the outer sheath 504 and the suction force applied through the space 506 move the target tissue into the space 506. Once a desired tissue sample has been collected in the space 506, the outer sheath 506 is moved distally over the needle 502. The beveled edge 521 then cuts the tissue sample from the surrounding tissue such that the device 500 may be removed from the patient body with the tissue sample collected therein.

It will be apparent to those skilled in the art that variations can be made in the structure and methodology of the present disclosure, without departing from the scope of the disclosure. Thus, it is intended that the present disclosure cover the modifications and variations of this disclosure provided that they come within the scope of the appended claims and their equivalents.

1-15. (canceled)

16. A device for collecting a tissue sample, comprising:
an outer sheath extending longitudinally from a proximal end to a distal end and including a lumen extending therethrough;
a needle movably housed within the outer sheath, the needle extending longitudinally from a proximal end to a serrated distal end and including a channel extending therethrough, the needle being longitudinally movable relative to the outer sheath between an insertion configuration, in which the distal end of the needle is proximal of the distal end of the outer sheath, and a tissue collecting configuration, in which the distal end of the needle extends distally past the distal end of the outer sheath to penetrate target tissue and to collect a tissue sample in the channel; and
a drive mechanism rotating a distal portion of the needle about a longitudinal axis thereof as the needle is moved between the insertion configuration and the tissue collecting configuration.

17. The device of claim 16, wherein the drive mechanism includes a helical structure extending about the distal portion of the needle and a corresponding helical structure along an interior surface of a drive collar connected to the distal end of the outer sheath, the helical structure of the needle and the corresponding helical structure of the drive collar engaging one another such that, when the needle is moved longitudinally relative to the outer sheath, the needle is rotated relative to the outer sheath.

18. The device of claim 16, wherein the needle includes a barb extending laterally into the channel to grip tissue received therewithin.

19. The device of claim 18, wherein the barb is a tab formed by cutting through a wall of the needle and bending the tab laterally into the channel.

20. The device of claim 16, wherein the drive mechanism includes a first ratchet mechanism and a second ratchet mechanism coupling a proximal portion and a distal portion of the needle, the first ratchet mechanism including a rod extending distally from the proximal portion to engage ratchet teeth along an interior surface of the distal portion, and the second ratchet mechanism including a set of teeth about each of a distal end of the proximal portion and a proximal end of the distal portion such that teeth of the first and second ratchet mechanisms are alternatingly engaged to rotate the distal portion relative to the proximal portion.

21. The device of claim 20, wherein the first and second ratchet mechanisms are alternatingly engaged via a linear oscillation of the proximal portion.

22. The device of claim 16, further comprising a stylet including a protrusion extending laterally therefrom.

23. The device of claim 22, wherein the drive mechanism includes a ratchet mechanism coupling a proximal portion and a distal portion of the needle, the ratchet mechanism including a groove extending along an interior surface of the distal portion of the needle, the protrusion of the stylet engaging ratchet teeth along the groove so that a linear oscillation of the stylet rotates the distal portion relative to the proximal portion.

24. The device of claim 16, wherein the needle includes a proximal portion and a distal portion connected to one another via a pivot joint controlled via a plurality of control wires to pivot the distal portion relative to the proximal portion.

25. The device of claim 16, wherein the needle includes a plurality of holes extending laterally through a distal portion thereof for permitting fluid to leak therethrough as tissue is being cut.

26. The device of claim 16, further comprising a suction source applying a suction force through the channel of the needle to draw tissue thereinto.

27. A device for collecting a tissue sample, comprising:
an outer sheath extending longitudinally from a proximal end to a distal end and including a lumen extending therethrough;
a needle slidably housed within the outer sheath, the needle extending longitudinally from a proximal end to a tapered distal end, the needle longitudinally movable relative to the outer sheath between a retracted configuration and a tissue collecting configuration in which the needle is moved distally relative to the outer sheath.

28. The device of claim 27, wherein a tissue sample is collected in a space between an interior surface of the lumen of the outer sheath and an exterior surface of the needle.

29. The device of claim 28, wherein a suction force is applied through the space to draw tissue thereinto.

30. The device of claim 27, wherein the needle is moved between the retracted and tissue collecting configurations via a drive mechanism including a first cam attached to the proximal end of the needle, an second cam housed within a proximal end of the outer sheath and a spring element biasing the device toward the retracted configuration.

31. A method for collecting a tissue sample, comprising:
inserting a tissue collecting device to a target tissue within a patient body via a working channel of an endoscope, in an insertion configuration in which a needle is housed within an outer sheath so that a distal end of the needle is proximal a distal end of the outer sheath; and
moving the needle distally relative to the outer sheath to a tissue collecting configuration in which the distal end of the needle extends distally beyond the distal end of the outer sheath to be inserted into the target tissue, wherein the needle rotates about a longitudinal axis thereof via a drive mechanism as the needle is advanced distally out of the sheath such that a serrated distal edge of the needle cores a tissue sample from the target tissue collecting the tissue sample therewithin.

32. The method of claim 31, further comprising moving the needle from the tissue collecting position to the retracted position to withdraw the device from the patient body.

33. The method of claim 31, wherein the drive mechanism includes a helical structure extending about a distal portion thereof and a corresponding helical structure along an interior surface of a drive collar connected to the distal end of the outer sheath, the helical structure of the needle and the corresponding helical of the outer sheath engaging one another such that, when the needle is moved longitudinally relative to the outer sheath, the needle is rotated relative to the outer sheath.

34. The method of claim 31, wherein the drive mechanism includes a first ratchet mechanism and a second ratchet mechanism coupling a proximal portion and a distal portion of the needle, the first ratchet mechanism including a rod extending distally from the proximal portion to engage ratchet teeth along an interior surface of the distal portion, and the second ratchet mechanism including a set of teeth about each of a distal end of the proximal portion and a proximal end of the distal portion such that teeth of the first and second ratchet mechanisms are alternatingly engaged to rotate the distal portion relative to the proximal portion.

35. The method of claim 31, wherein the drive mechanism includes a ratchet mechanism coupling a proximal portion and a distal portion of the needle, the ratchet mechanism including a groove extending along an interior surface of the distal portion of the needle to receive a protrusion extending laterally from a portion of a stylet received within the channel, the protrusion of the stylet engaging ratchet teeth along the groove so that a linear oscillation of the stylet rotates the distal portion relative to the proximal portion.
